(19) United States
(12) Patent Application Publication
(10) Pub. No.: US 2009/0305380 A1
(43) Pub. Date: Dec. 10, 2009

(54) ELECTROPORATION OF ADHERENT CELLS WITH AN ARRAY OF CLOSELY SPACED ELECTRODES

(75) Inventor: Charles W. Ragsdale, Concord, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: BIO-RAD LABORATORIES, INC., Hercules, CA (US)

(21) Appl. No.: 12/437,760

(22) Filed: May 8, 2009

Related U.S. Application Data
(60) Provisional application No. 61/052,728, filed on May 13, 2008.

(51) Int. Cl. C12N 13/00; C12M 1/42

(52) U.S. Cl. 435/173.6; 435/285.2

(57) ABSTRACT

Adherent cells and other membranous structures that are immobilized on a solid surface are transfected by electroporation in which the electric field is produced by a array of closely spaced electrodes positioned above the surface. Each electrode is substantially smaller in at least one lateral dimension than the dimensions of a single cell, and the electrodes in each pair are spaced apart by distances selected such that that a maximum of one cell will reside within the field produced by each pair, and the distance of the electrodes above the surface to which the cells are adherent is small enough to place the cell within the resulting electric field and yet great enough to avoid contact of the electrodes with the cell membrane.

### CROSS-REFERENCE TO RELATED APPLICATION

This application claims the benefit of U.S. Provisional Patent Application No. 61/052,728, filed May 13, 2008, the contents of which are incorporated herein by reference.

### BACKGROUND OF THE INVENTION

1. Field of the Invention

This invention lies in the field of transfection, the process by which exogenous molecular species are inserted into membranous structures by rendering the membrane permeable on a transient basis while the structures are in contact with a liquid solution of the species, thereby allowing the species to pass through the membrane.

2. Description of the Prior Art

Certain biologic and biochemical techniques involve the introduction of exogenous species into biological cells. The process of introduction is termed transfection, and transfections of high efficiency are those in which the exogenous species has successfully entered a high proportion of the cells of the population being treated and in which the viability of the cells has either been maintained throughout or restored after the procedure. Of the various transfection techniques, electroporation, which is the use of an electric field to cause a transient permeabilization of the cell membrane, has received the most attention. Transfection has been performed both on cells that are suspended in a buffer solution and on adherent cells, i.e, cells that are immobilized on a solid surface which is often the surface on which the cells have been grown. Achieving high efficiency is a continuing challenge in all forms of electroporation, but even more so in the electroporation of adherent cells. Disclosures of the electroporation of adherent cells are found in the following published documents:

Jarvis et al., U.S. Pat. No. 6,897,069 B1, issued May 24, 2005

Lee et al., United States Patent Application Publication No. US 2007/0155016 A1, published Jul. 5, 2007

Vassanelli et al., United States Patent Application Publication No. US 2007/0115015 A1, published Jul. 5, 2007

Huang et al., United States Patent Application Publication No. US 2005/070510 A1, published Aug. 4, 2005

Acker, United States Patent Application Publication No. US 2004/0029240 A1, published Feb. 12, 2004

Zimmerman et al., United States Patent Application Publication No. US 2003/0148524 A1, published Aug. 7, 2003

Meyer, U.S. Pat. No. 6,261,815 B1, issued Jul. 17, 2001, issued Jul. 17, 2001

Korenstein et al., U.S. Pat. No. 5,964,726, issued Oct. 12, 1999

Casnig, U.S. Pat. No. 5,134,070, issued Jul. 28, 1992

Raptis, U.S. Pat. No. 6,001,617, issued Dec. 124, 1999

While the documents in the above list present a variety of approaches to improving the efficiency and uniformity of transfection, these qualities remain elusive and are a continuing goal. In addition to the difficulties presented by the adherent nature of the cells, transfection efficiency also suffers from the variation to which different membranous structures are exposed to the same electric field in any electroporation procedure. When the structures are biological cells, for example, a typical cell population contains cells of different degrees of maturity or cells at different stages of their life cycles. A cell population of a single cell line can thus include cells of different sizes. The voltage across a single cell will be proportional to the cell diameter, and thus for a given field intensity, the voltage difference across a small cell will be lower than that across a large cell. A voltage difference that is too low will fail to render the cell wall sufficiently porous to allow the molecules to penetrate the wall, while a voltage that is too high will cause lysis of the cell.

### SUMMARY OF THE INVENTION

The present invention resides in a method and apparatus for electroporation of adherent cells or other immobilized membranous structures, in which individual cells are exposed to a highly focused electric field that does not vary with the cell size. In accordance with this invention, an array of electric fields, produced by an array of pairs of closely spaced electrodes distributed within a plane that is positioned substantially parallel to the solid surface on which the adherent cells reside. The electrode array is close enough to the solid surface that the electric fields intersect the surface without the electrodes contacting the cells. The electrodes within each adjacent pair are close enough to each other that no more than one biological cell resides within length of the shortest distance between the electrodes. The electrodes are either dot-form electrodes (i.e., electrodes in the form of dots) or elongated strip electrodes such as exposed lengths of wire. In the case of dot-form electrodes, the shortest distance referred to above is the distance between the two adjacent and oppositely polarized dots. In the case of elongated wire or strip electrodes, the shortest distance is the distance along a line perpendicular to the electrodes themselves. By stating that no more than one cell resides within the shortest distance between the two electrodes, it is meant herein that the electrodes are close enough that the distance between them is equal to or less than the width of a single cell, or that if the distance is greater than the width of a single cell, the cells themselves are spaced far enough apart on the solid surface that no more than one cell will reside within the distance separating the electrodes. This is true even though the electrodes and the cells are in different, although substantially parallel, planes, i.e., the distance between the electrodes is compared to the cell width and/or the spacing of the cells by projection of the electrodes into the plane of the cells, or vice versa. In most cases, the cells will be either equal to or larger in diameter than the shortest distance between the adjacent electrodes. Cells of relatively large diameters will therefore be exposed to electric fields from more than one pair of adjacent electrodes, including pairs that share a common electrode.

In embodiments where the electrodes are a series of parallel lines or traces, the widths of the lines or traces are in the micron range, substantially less than the diameters of the cells, and lines of positive polarity will preferably alternate with lines of negative polarity. Electroporation of the cells across a two-dimensional area is readily achieved either by energizing all of the line electrodes simultaneously (with positively charged electrodes alternating with negatively charged electrodes) or by energizing adjacent pairs of line electrodes in sequence. In embodiments utilizing dot electrodes, the dots have diameters in the micron range, substantially less than the diameters of the cells, and are preferably arranged in a straight line or in two or more parallel straight lines, with polarities alternating among the dots in a single line or between the dots of adjacent parallel lines, i.e., the dots of one line having a polarity opposite that of the dots in an adjacent line. In all cases, electroporation of the cells across a two-dimensional area is readily achieved either by energizing all electrodes simultaneously or by energizing adjacent pairs in sequence. With a single line of dot-form electrodes or a narrow strip of parallel lines of dot-form electrodes, electroporation of the cells across a two-dimensional area is achieved by mounting the electrodes on a movable support and traversing the area with the support.

These and other objects, features, and advantages of the invention will be more apparent from the description that follows.

### BRIEF DESCRIPTION OF THE DRAWINGS

FIG. 1 is an end view of an electrode support for use in certain embodiments of the present invention.

FIG. 2 is a side view of the electrode support of FIG. 1.

FIG. 3 is a top view of a surface for adherent cells and a mobile electrode support in accordance with certain embodiments of the invention.

FIG. 4 is a top view of an alternative surface for adherent cells and a mobile electrode support in accordance with certain other embodiments of the invention.

FIG. 5 is a perspective view of an electroporation apparatus that utilizes a two-dimensional array of parallel-line electrodes in accordance with certain embodiments of the invention.

FIG. 6 is a second perspective view of the apparatus of FIG. 5 with parts separated to show their internal surfaces.

FIG. 7 is a view of the underside of the electrode support serving as one of the parts of the apparatus of FIGS. 5 and 6.

FIG. 8 is a cross section of a vessel containing the electroporation apparatus of FIGS. 5, 6, and 7.

### DETAILED DESCRIPTION OF THE INVENTION AND PREFERRED EMBODIMENTS

The most typical adherent cells are biological cells that are adherent to the surface on which they are grown. The concerns that apply to such cells can also arise in the electroporation of cells that are immobilized for other purposes or that have become immobilized by other means. The present invention is thus directed to the electroporation of adherent membranous structures in general, including such structures as vesicles and liposomes in addition to cells. The terms “cell” and “biological cell” will be used herein for convenience to collectively denote all such membranous structures. Examples of the species, referred to herein as “exogenous species” or “transfecting species,” that will pass through the membranes of these cells during the electroporation, are nucleic acids including DNA, RNA, plasmids, and genes and gene fragments, and proteins, pharmaceuticals, and enzyme cofactors. Further examples of exogenous species will be apparent to those skilled in the art.

The solid surface to which the cells adhere can be the surface of any material that is capable of serving as an immobilizing support for the cells. Such surfaces can be the surfaces of glass, polycarbonate, polystyrene, polyvinyl, polyethylene, polypropylene, or a variety of other materials known to cell biologists. Microporous membranes used in membrane-based cell culture can also be used. Examples are membranes of hydrophilic poly(tetrafluoroethylene), cellulose esters, polycarbonate, and polyethylene terephthalate. A membrane that is otherwise flexible can made flat and rigid by placing the membrane over a support such as a flat screen or a block of solid glass or polymeric material. Adherence of the cells to the surface can be achieved by conventional means, including the inherent adherence when the cells are grown on the surface, as well as adherence through immunological or affinity-type binding, electrostatic attraction, and covalent coupling.

For embodiments in which the electrodes are dots, referred to herein as “dot-form electrodes,” each electrode can be formed in a variety of ways. One example is by passing an electric wire through the bore of a glass pipet or microcapillary such that the wire is either exposed at the open end of the pipet or microcapillary or protrudes a short distance from the open end. Another example is by plating an electrical trace on an electrically insulating block or chip, preferably a block or chip with a sharply angled edge over which the trace passes, using conventional methods such as those employed in semiconductor manufacture. Once formed, the trace can be insulated by conventional masking material at all points except at the edge where the exposed trace serves as the dot-form electrode. Parallel line electrodes are also readily formed by conventional semiconductor manufacturing methods.

When a series of dot-form electrodes is used, the electrodes are preferably arranged in a straight line or in two or more parallel straight lines. Since the adherent cells are typically grown on a flat surface, and preferably a surface that is optically flat, the straight line of electrodes allows the electrodes to be positioned at a uniform height above the surface. The flat surface affords the cells their best opportunity for growth, for interaction among neighboring cells, and for uniform exposure to the electric fields. The straight line of dot-form electrodes is convenient for sweeping a two-dimensional area of cells with the electrodes.

The ability of the electrodes to form consistent electric fields for substantially all cells within the influence of the electrodes regardless of cell size is achieved by the narrowness or small diameter of the exposed surface of an individual electrode, the spacing between the adjacent electrodes, and the height of the electrodes above the surface on which the cells reside. These and other dimensions can vary with the nature of the cells, i.e., whether they be biological cells of various sources and cell lines, or liposomes, vesicles, or other membranous structures. Nevertheless, and particularly in the case of biological cells whose diameters are within the range of about 10 microns to about 20 microns, best results will be achieved in most cases with electrodes whose exposed surfaces are about 3 microns to about 20 microns in width or diameter, preferably about 5 microns to about 10 microns, and most preferably about 8 microns. For best results as well, the spacing between adjacent electrodes is about 20 microns to about 75 microns, preferably about 30 microns to about 50 microns, and most preferably about 40 microns. Best results are also achieved in most cases when the height of the electrodes above the surface on which the cells reside is from about 25 microns to about 100 microns, preferably from about 25 microns to about 50 microns, and most preferably about 40 microns. This height can be set by incorporating spacing legs, ridges, piers, or the like into the structure of the support on which the electrodes are mounted.

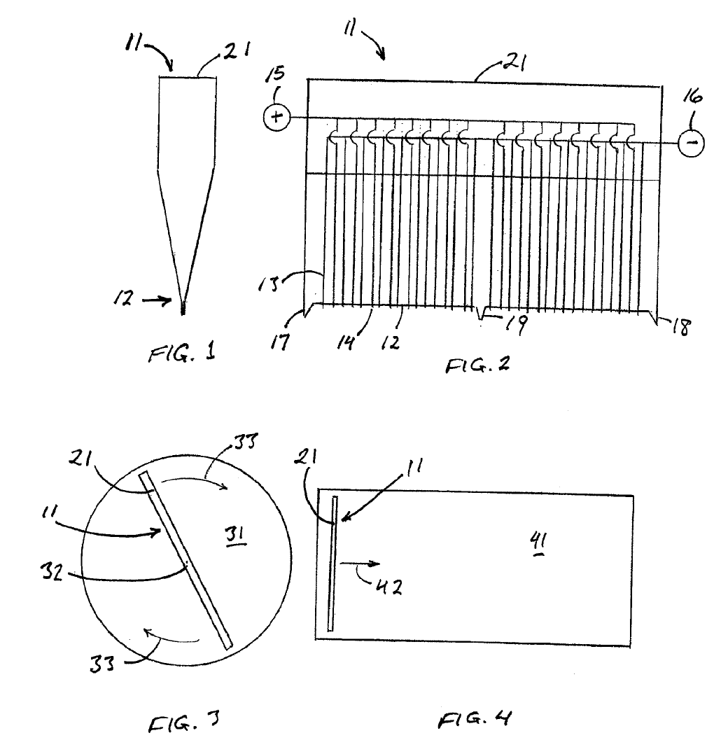

An example of a linear array of dot-form electrodes is shown in FIGS. 1, 2, 3, and 4. The electrodes in this example are positioned along the sharp edge of a wedge-shaped block. FIG. 1 is an end view of the block 11, with the sharp edge 12 shown at the bottom. FIG. 2 is a side view of the block, again with the sharp edge 12 of the wedge at the bottom. The electrodes are formed by electrical traces 13 that are plated on the block surface in parallel lines extending down one face of the wedge, across the sharp edge 12, and up the other face. The traces are electrically insulated with masking material at all points along their lengths except at the edge 12 where the gaps in the masking form a line of dots 14. The traces are electrically connected in alternating fashion, so that odd-numbered traces can be connected to one pole 15 of a power source and even-numbered traces to the other pole 16. At the ends of the sharp edge 12 of the block are legs 17, 18, with an additional leg 19 at the center of the edge. These legs, which are of equal length and are extensions of the block 11 or of the mask layer, contact the surface on which the cells reside to fix the height of the dot electrodes 12 above the surface with the cells in between.

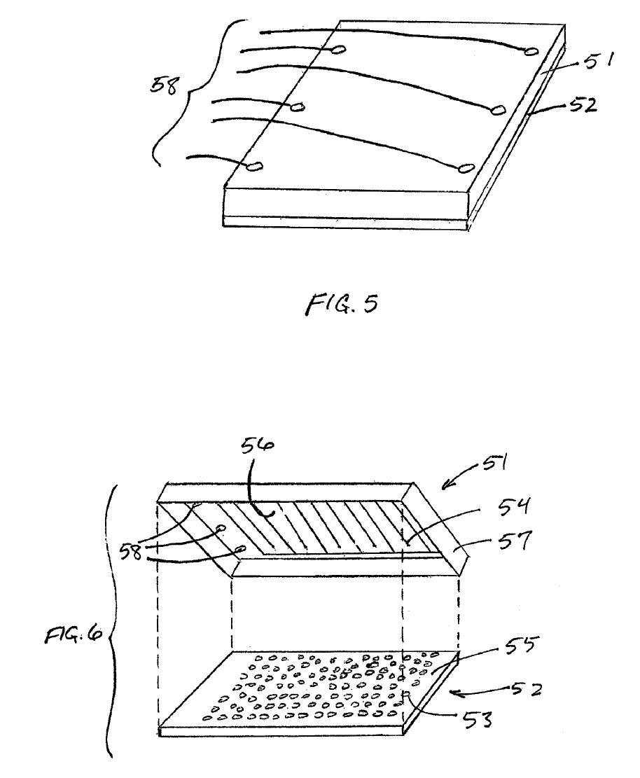

The scanning of a two-dimensional surface (on which the cells reside) with the block-supported electrodes is demonstrated in FIGS. 3 and 4 which offer top views of two surfaces, respectively. FIGS. 3 and 4 also show the top edge 21 of the block and the movement of the block. The surface 31 in FIG. 3 is a circular surface, and the block 11 scans the surface by rotating about its center 32, as indicated by the arrows 33. The surface 41 in FIG. 4 is a square or rectangular surface which the block 11 scans by moving laterally, in the direction of the arrow 42. Movement of the block in both cases is achieved by conventional means, such as a stepper motor or a dc motor.

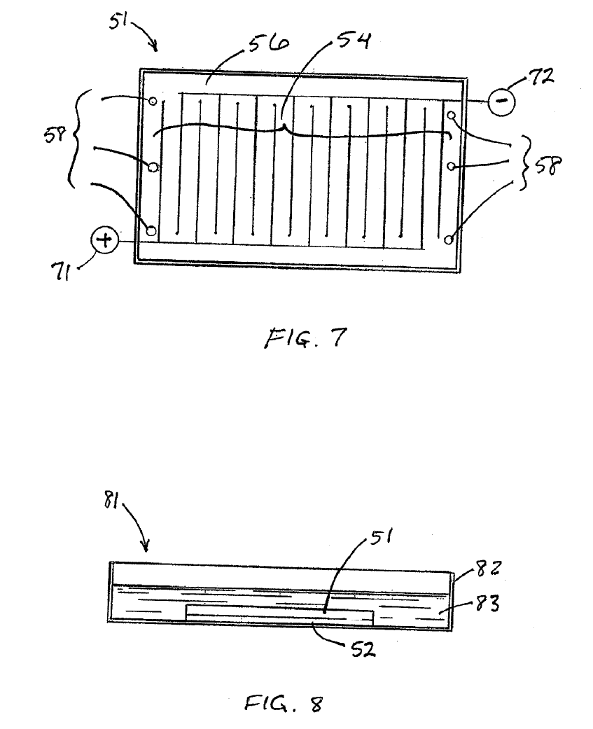

An example of a two-dimensional array of parallel-line electrodes is shown in FIGS. 5, 6, 7, and 8. FIG. 5 shows the combination of the electrode block 51 and a cell plate 52 in an assembled structure. Both the cells and the electrodes are internal to the assembled structure and therefore not visible in this view. FIG. 6 shows the block 51 and cell plate 52 separated (with exaggerated dimensions to more clearly illustrate the component parts), so that both the cells 53 and the electrodes 54 are visible. The cells 53 are grown on the optically flat surface 55 of the cell plate, and the electrodes 54 are plated onto the flat undersurface 56 of the electrode block. Optical flatness is not a requirement for the undersurface 56, despite the optical flatness of the cell plate surface 55, but the closer the surface is to optical flatness the more effectively the system will function in achieving uniform electroporation of the cells. Surrounding the flat undersurface 56 on which the line electrodes are plated is a ridge or raised edge 57 which, when the block 51 is pressed against the cell plate 52, will contact the cell plate surface 55 outside of the area occupied by the cells 53 and set the electrodes 54 at the desired distance above the plate surface 55 and hence the cells 53. To allow the space between the electrodes 54 and the cells 53 to be filled with the solution of the transfecting species that will enter the cells and to allow free movement of the solution through the space, the electrode block 51 contains a series of holes 58 (also visible in FIG. 5). The line electrodes 54 are most clearly seen in FIG. 7, which is a plan view of the undersurface 56 of the electrode block 51. The line electrodes are connected to a positive pole 71 and a negative pole 72 of a power source in alternating manner. Line electrodes of positive polarity will thus alternate with line electrodes of negative polarity. Finally, FIG. 8 shows an electroporation cell 81 consisting of a reservoir 82 in which the assembled structure of the electrode block 51 and a cell plate 52 as depicted in FIG. 5 are placed, together with the solution 83 of the transfecting species, the block and cell plate assembly fully immersed in the solution.

In each of the configurations shown in the drawings, whether of dot-form electrodes or line electrodes, all of the electrodes can be energized or pulsed simultaneously, or adjacent pairs can be energized in succession along the length of the array. When adjacent pairs are individually energized, each pair will produce an electric field that will encompass cells on the portion of the cell plate surface that is between the electrodes in the pair, and the electrodes are spaced such that no more than a single cell will reside within the field of a single pair of electrodes.

Power sources and energization protocols that are known in the electroporation art can be used. The use of a pulsed electric field is preferred, and the pulse duration will typically be within the range of about 1 microsecond to about 1 second, preferably from about 50 microseconds to about 10 milliseconds.

In the claim or claims appended hereto, the term “a” or “an” is intended to mean “one or more.” The term “comprise” and variations thereof such as “comprises” and “comprising,” when preceding the recitation of a step or an element, are intended to mean that the addition of further steps or elements is optional and not excluded. All patents, patent applications, and other published reference materials cited in this specification are hereby incorporated herein by reference in their entirety. Any discrepancy between any reference material cited herein and an explicit teaching of this specification is intended to be resolved in favor of the teaching in this specification. This includes any discrepancy between an art-understood definition of a word or phrase and a definition explicitly provided in this specification of the same word or phrase.

What is clamed is:

1. Apparatus for transfection of adherent biological cells immobilized on a solid surface, said apparatus comprising:
a support for said solid surface; and
an array of pairs of adjacent electrodes residing within a plane that is substantially parallel to said solid surface and sufficiently close to said solid surface such that each said pair of adjacent electrodes when energized produces an electric field that intersects said solid surface, the electrodes within each pair of adjacent electrodes being sufficiently close to each other that no more than one of said biological cells resides within the shortest distance between said electrodes of each pair of adjacent electrodes.

2. The apparatus of claim 1 wherein said electric fields produced by said array of pairs of adjacent electrodes collectively intersect a portion of said solid surface that is less than the full area of said solid surface, and said apparatus further comprises means for translating said array within said plane to traverse said full area.

3. The apparatus of claim 1 wherein said array of pairs of adjacent electrodes comprises a linear array of dot electrodes, and said apparatus further comprises means for translating said linear array across said solid surface.

4. The apparatus of claim 3 wherein said means for translating are means for rotating said linear array over a circular area of said solid surface.

5. The apparatus of claim 3 wherein said means for translating are means for conveying said linear array in a direction transverse to said linear array.

6. The apparatus of claim 1 wherein array of pairs of adjacent electrodes is a two-dimensional array of parallel line electrodes.

7. The apparatus of claim 1 wherein said electrodes within each of said pairs of adjacent electrodes are spaced apart by a distance of about 20 microns to about 75 microns.

8. The apparatus of claim 1 wherein said electrodes within each of said pairs of adjacent electrodes are spaced apart by a distance of about 30 microns to about 50 microns.

9. The apparatus of claim 1 wherein said plane containing said array of pairs of adjacent electrodes is spaced apart from said solid surface by a distance of about 25 microns to about 100 microns.

10. The apparatus of claim 1 wherein said plane containing said array of pairs of adjacent electrodes is spaced apart from said solid surface by a distance of about 25 microns to about 50 microns.

11. A process for the transfection of a population of adherent biological cells immobilized on a solid surface, said process comprising:
placing said solid surface parallel to an array of pairs of adjacent electrodes and sufficiently close to said array that each said pair of adjacent electrodes when energized produces an electric field that intersects said solid surface, without contact between said electrodes and said cells, the electrodes within each pair of adjacent electrodes being sufficiently close to each other that no more than one of said biological cells resides within the shortest distance between said electrodes of each pair of adjacent electrodes; and
with said solid surface so placed and while said solid surface is immersed in a liquid solution of a transfecting species, energizing said electrodes to cause transfection of said biological cells with said transfecting species.

12. The process of claim 11 wherein said biological cells are distributed over an area whose dimensions exceed the dimensions of said array of pairs of adjacent electrodes, said process further comprising moving said array of pairs of adjacent electrodes within a plane parallel to said solid surface to encompass said entire area.

13. The process of claim 12 wherein said area is a circular area, said array of pairs of adjacent electrodes comprises a linear array of dot electrodes, and said process further comprises rotating said linear array over said circular area.

14. The process of claim 12 wherein said array of pairs of adjacent electrodes comprises a linear array of dot electrodes, and said process further comprises conveying said linear array in a direction transverse to said linear array.

15. The process of claim 11 wherein said array of pairs of adjacent electrodes is a two-dimensional array of parallel line electrodes.

16. The process of claim 11 wherein said electrodes within each of said pairs of adjacent electrodes are spaced apart by a distance of about 20 microns to about 75 microns.

17. The process of claim 11 wherein said electrodes within each of said pairs of adjacent electrodes are spaced apart by a distance of about 30 microns to about 50 microns.

18. The process of claim 11 wherein said array of pairs of adjacent electrodes reside in a plane spaced apart from said solid surface by a distance of about 25 microns to about 100 microns.

19. The process of claim 11 wherein said array of pairs of adjacent electrodes reside in a plane spaced apart from said solid surface by a distance of about 25 microns to about 50 microns.
